(19) United States
(12) Patent Application Publication
(10) Pub. No.: US 2013/0030299 A1
(43) Pub. Date: Jan. 31, 2013

(54) ULTRASOUND SYSTEM AND METHOD FOR CORRECTING DOPPLER ANGLE

(71) Applicant: Dae Young KIM, Seoul (KR)

(72) Inventor: Dae Young KIM, Seoul (KR)

(73) Assignee: SAMSUNG MEDISON CO., LTD.

(21) Appl. No.: 13/559,389

(22) Filed: Jul. 26, 2012

(30) Foreign Application Priority Data
Jul. 26, 2011 (KR) 10-2011-0074257

(51) Int. Cl. A61B 8/00

(52) U.S. Cl. 600/453

(57) ABSTRACT

There are provided embodiments for measuring an inclination angle of an ultrasound probe to correct a Doppler angle in real time. In one embodiment, by way of non-limiting example, an ultrasound system comprises: an inclination measuring unit configured to measure an inclination angle of an ultrasound probe at a predetermined cycle to form measuring information including the inclination angle, wherein the inclination measuring unit is mounted inside or outside of the ultrasound probe; and a processing unit configured to calculate a Doppler angle correction value corresponding to the inclination angle based on the measuring information and calculate a corrected Doppler angle based on the Doppler angle correction value.

### CROSS-REFERENCE TO RELATED APPLICATIONS

The present application claims priority from Korean Patent Application No. 10-2011-0074257 filed on Jul. 26, 2011, the entire subject matter of which is incorporated herein by reference.

### TECHNICAL FIELD

The present invention generally relates to ultrasound systems, and more particularly to an ultrasound system and a method for measuring an inclination angle of an ultrasound probe to correct a Doppler angle in real time.

### BACKGROUND

An ultrasound system is widely used in the medical applications for acquiring information of inner parts of living bodies due to its non-invasive and non-destructive nature. The ultrasound system can provide high dimensional real-time ultrasound images of the inner parts of the living bodies without any surgical operation. Thus, the ultrasound system is very important in medical applications.

The ultrasound system may transmit ultrasound signals to the living bodies including target objects (e.g., blood flows, hearts, etc.) via an ultrasound probe. It may then receive ultrasound echo signals reflected from the living bodies to thereby provide ultrasound images. Especially, the ultrasound system may provide Doppler mode images by using the Doppler effect. In the color Doppler mode images, velocities of the target objects may be represented by Doppler spectrums or colors. The Doppler mode images may include Doppler spectrum images or color Doppler images.

The ultrasound probe may transmit the ultrasound signals and receive the echo signals. A front end of the ultrasound system may convert the received echo signals into digital signals. Receive-focused signals may be formed by receive (Rx) focusing the digital signals. The receive-focused signals may be bandwidth-converted by using a mixer and converted into in-phase/quadrature (IQ) signals by using appropriate decimation. The IQ signals are referred to as baseband IQ signals.

The IQ signals are represented as equation 1 provided below.

X_{IQ}=C+F+N (1)

wherein “X_{IQ}” denotes the IQ signal, “C” denotes the clutter signal produced by tissues of the target objects, “F” denotes the flow signal produced by the blood flow, and “N” denotes the noise produced by the ultrasound system and outside of the ultrasound system.

A user may acquire velocities of the blood flow by using the ultrasound system. The Doppler formula is used to calculate the velocities of the blood flow. The Doppler formula is represented as equation 2 provided below.

$${f}_{D} = \frac{2 {f}_{0} v cos θ}{c} \qquad ( 2 )$$

wherein “f_{D}” denotes the Doppler frequency, “f₀” denotes an ultrasound frequency transmitted from the ultrasound probe, “ν” denotes the velocity of the blood flow, “C” denotes the velocities of sound, and “θ” denotes an angle between a moving path of the blood flow and a beam direction transmitted from the ultrasound probe. The “θ” may be referred to as the Doppler angle.

Equation 2 may be reformulated in terms of “ν” (e.g., the velocity of the blood flow) as equation 3 provided below.

$$v = \frac{{cf}_{D}}{2 {f}_{0} cos θ} \qquad ( 3 )$$

In equation 3, the Doppler angle “θ” is an important variable for determining the velocities of the blood flow.

Conventionally, the user sets the Doppler angle by using a sample-volume angle setting function in the ultrasound system. That is, the user inputs the angle between the moving path of the blood flow and the beam direction transmitted from the ultrasound probe. As a result, there is a disadvantage since it is required to frequently set the sample-volume angle during observation to adjust a difference of the angle between the moving path of the blood flow and the beam direction transmitted from the ultrasound probe, wherein the difference of the angle is caused by tilting of the ultrasound probe.

### SUMMARY

There is provided an embodiment for measuring an inclination angle of an ultrasound probe by using an inclination measuring unit mounted inside or outside of the ultrasound probe and correcting a Doppler angle based on the inclination angle in real time.

In one embodiment, by way of non-limiting example, an ultrasound system may include: an inclination measuring unit configured to measure an inclination angle of an ultrasound probe at a predetermined cycle to form measuring information including the inclination angle, wherein the inclination measuring unit is mounted inside or outside of the ultrasound probe; and a processing unit configured to calculate a Doppler angle correction value corresponding to the inclination angle based on the measuring information and calculate a corrected Doppler angle based on the Doppler angle correction value.

In another embodiment, a method of correcting a Doppler angle may comprise: measuring an inclination angle of an ultrasound probe at a predetermined cycle to form measuring information including the probe angle; calculating a Doppler angle correction value corresponding to the inclination angle based on the measuring information; and calculating a corrected Doppler angle based on the Doppler angle correction value.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a flow chart showing a process of correcting a Doppler angle to form a Doppler mode image.

### DETAILED DESCRIPTION

This detailed description is provided with reference to the accompanying drawings. One of ordinary skill in the art may realize that the following description is illustrative only and is not in any way limiting. Other embodiments of the present invention may readily suggest themselves to such skilled persons having the benefit of this disclosure.

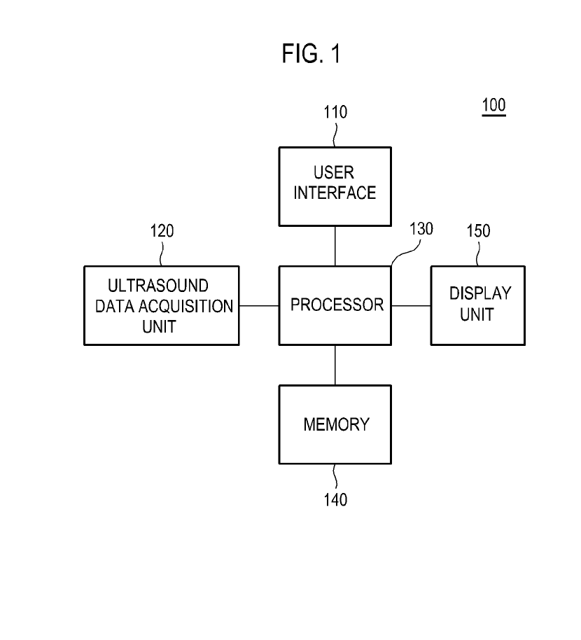

FIG. 1 is a block diagram showing an illustrative embodiment of an ultrasound system 100. The ultrasound system 100 may include a user interface 110, an ultrasound data acquisition unit 120, a processor 130, a memory 140 and a display unit 150.

The user interface 110 may be configured to receive input information from a user. In one embodiment, the input information may include information for setting a region of interest on the B-mode image. The region of interest may include a sample volume for acquiring Doppler spectrum images or a color box for acquiring color Doppler images. However, it should be noted herein that the region of interest may not be limited thereto.

The ultrasound data acquisition unit 120 may be configured to transmit ultrasound signals to a living body. The living body may include target objects (e.g., blood flows, hearts, vascular, etc.). The ultrasound data acquisition unit 120 may be further configured to receive ultrasound signals (i.e., ultrasound echo signals) reflected from the living body to acquire ultrasound data.

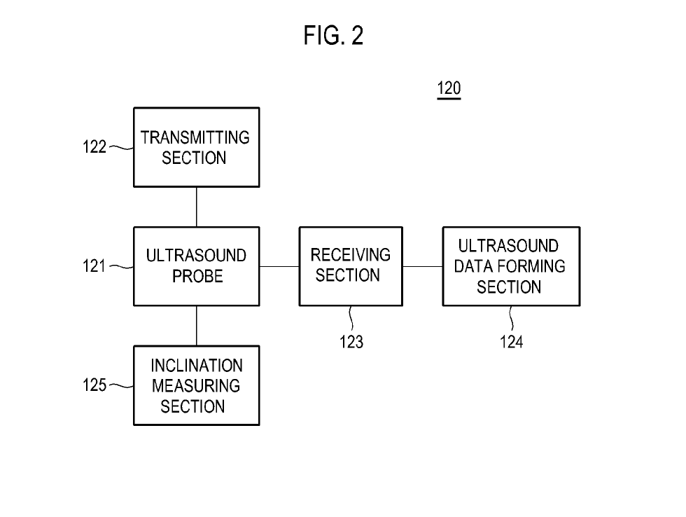

FIG. 2 is a block diagram showing an illustrative embodiment of an ultrasound data acquisition unit 120. Referring to FIG. 2, the ultrasound data acquisition unit 120 may include an ultrasound probe 121, a transmitting section 122, a receiving section 123, an ultrasound data forming section 124 and an inclination measuring section 125.

The ultrasound probe 121 may include a plurality of transducer elements (not shown) for reciprocally converting between electrical signals and the ultrasound signals. The ultrasound probe 121 may be configured to transmit the ultrasound signals to the living body and receive the ultrasound echo signals reflected from the living body to output electrical signals (hereinafter, referred to as “reception signals”). The reception signals may be analog signals.

The transmitting section 122 may be configured to control the transmission of the ultrasound signals. The transmitting section 122 may be configured to generate electrical signals (hereinafter, referred to as “transmission signals”) for acquiring an ultrasound image in consideration of transducer elements and focal points.

In one embodiment, the transmitting section 122 may be configured to generate first transmission signals for acquiring a B-mode image in consideration of the transducer elements and the focal points. Thus, the ultrasound probe 121 may be configured to convert the first transmission signals provided from the transmitting section 122 into the ultrasound signals, transmit the ultrasound signals to the living body, and receive the ultrasound echo signals reflected from the living body to output first reception signals. The transmitting section 122 may be further configured to generate second transmission signals for acquiring a Doppler mode image corresponding to the region of interest in consideration the transducer elements and the focal points. Thus, the ultrasound probe 121 may be configured to convert the second transmission signals provided from the transmitting section 122 into the ultrasound signals, transmit the ultrasound signals to the living body, and receive the ultrasound echo signals reflected from the living body to output second reception signals.

The receiving section 123 may be configured to perform an analog-digital conversion upon the reception signals provided from the ultrasound probe 121 to form digital signals. The receiving section 123 may be further configured to perform a reception beam-forming upon the digital signals in consideration of the transducer elements and the focal points to form reception-focused signals.

In one embodiment, the receiving section 123 may be configured to perform the analog-digital conversion upon the first reception signals provided from the ultrasound probe 121 to form first digital signals. The receiving section 123 may be further configured to perform the reception beam-forming upon the first digital signals in consideration of the transducer elements and the focal points to form first reception-focused signals. The receiving section 123 may be also configured to perform the analog-digital conversion upon the second reception signals provided from the ultrasound probe 121 to form second digital signals. The receiving section 123 may be additionally configured to perform the reception beam-forming upon the second digital signals in consideration of the transducer elements and the focal points to form second reception-focused signals.

The ultrasound data forming section 124 may be configured to form ultrasound data based on the reception-focused signals provided from the receiving section 123. The ultrasound data forming section 124 may be further configured to perform a signal process (e.g., gain control, etc) upon the reception-focused signals.

In one embodiment, the ultrasound data forming section 124 may be configured to form first ultrasound data corresponding to the B-mode image based on the first reception-focused signals provided from the receiving section 123. The first ultrasound data may include radio frequency data. However, it should be noted herein that the first ultrasound data may not be limited thereto. The ultrasound data forming section 124 may be further configured to form second ultrasound data corresponding to the region of interest (i.e., Doppler mode image) based on the second reception-focused signals provided from the receiving section 123.

The inclination measuring section 125 may be configured to measure an inclination angle of the ultrasound probe 121 (hereafter, referred to as a “probe angle”) at a predetermined cycle to thereby form measuring information including the probe angle. The predetermined cycle may represent a cycle for performing Doppler calculation, i.e., calculation of the blood flow velocities. However, it should be noted herein that the predetermined cycle may not be limited thereto. The inclination measuring section 125 may be mounted inside or outside of the ultrasound probe 121. However, it should be noted herein that the inclination measuring section 125 may not be limited thereto. The inclination measuring section 125 may be connected to the processor 130 in a wired or wireless manner. Any apparatus, which can measure the inclination angle of the ultrasound probe 121, may be adopted as the inclination measuring section 125. For example, the inclination measuring section 125 may include a gyroscope, an accelerometer and the like.

In one embodiment, the inclination measuring section 125 may be configured to start the measurement of the inclination angle of the ultrasound probe 121 according to the Doppler angle correction start under the control of the processor 130 to form first measuring information. Then, the inclination measuring section 125 may be configured to measure the inclination angle of the ultrasound probe 121 at the predetermined cycle to form second measuring information, third measuring information, . . . n^{th} measuring information. Furthermore, the inclination measuring section 125 may be configured to end the measurement of the inclination angle of the ultrasound probe 121 according to the Doppler angle correction end under the control of the processor 130.

In another embodiment, the inclination measuring section 125 may be configured to measure the inclination angle of the ultrasound probe 121 when the ultrasound probe 121 is operated (i.e., ultrasound probe 121 is activated) to form the measuring information. Furthermore, the inclination measuring section 125 may be configured to end the measurement of the inclination angle of the ultrasound probe 121 when the ultrasound probe 121 is not operated (i.e., ultrasound probe 121 is deactivated).

Referring back to FIG. 1, the processor 130 may be configured to form the ultrasound image based on the ultrasound data provided from the ultrasound data acquisition unit 120. The processor 130 may include a central processing unit, a microprocessor, a graphic processing unit and the like.

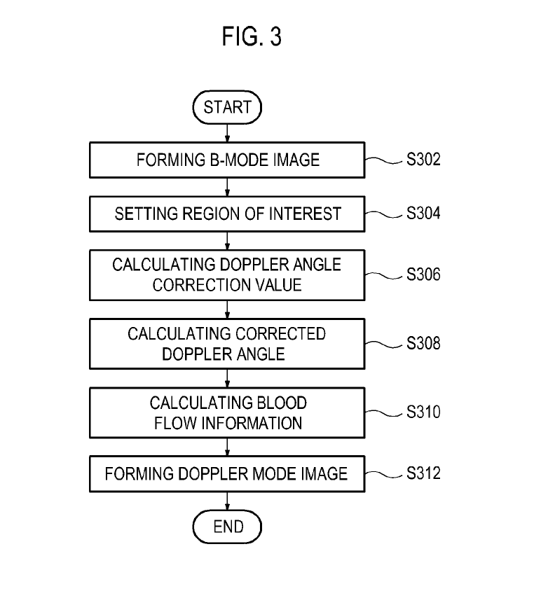

FIG. 3 is a flow chart showing a process of correcting a Doppler angle to form a Doppler mode image. The processor 130 may be configured to form the B-mode image based on the first ultrasound data provided from the ultrasound data acquisition unit 120 at step S302 in FIG. 3. The B-mode image may be displayed on the display unit 150. Thus, the user may set the region of interest on the B-mode image by using the user interface 110.

The processor may be configured to set the region of interest on the B-mode image based on the input information provided from the user interface 110 at step S304. Thus, the ultrasound data acquisition unit 120 may be configured to transmit the ultrasound signals to the living body and receive the ultrasound echo signals reflected from the living body to acquire the second ultrasound data corresponding to the region of interest.

The processor 130 may be configured to calculate a Doppler angle correction value based on the measurement information provided form the inclination measuring section 125 at step S306 in FIG. 3.

In one embodiment, the processor 130 may calculate the Doppler angle correction value by using the following equation:

Δφₙ=φₙ−φ₀ (4)

wherein φₙ represents the probe angle (hereinafter, referred to as “n^{th} probe angle”) of the ultrasound probe 121 at a present cycle (i.e., n^{th} cycle), φ₀ represents an initial probe angle of the ultrasound probe 121, and Δφₙ represents the Doppler angle correction value at the present cycle (i.e., angle variation between initial probe angle φ₀ and n^{th} probe angle φₙ). The initial probe angle may represent the inclination angle, which is initially measured by the inclination measuring section 125, when the second ultrasound data are acquired. However, it should be noted herein that the initial probe angle may not be limited thereto.

In another embodiment, the processor 130 may be configured to calculate the Doppler angle correction value as equation 5 provided below.

Δφₙ=φₙ−Δφₙ₋₁ (5)

In equation 5, φₙ represents the n^{th} probe angle of the ultrasound probe 121, Δφₙ₋₁ represents the Doppler angle correction value (hereinafter, referred to as “(n−1)^{th} Doppler angle correction value”) at a previous cycle (i.e., (n−1)^{th} cycle), and Δφₙ represents the Doppler angle correction value at the present cycle (i.e., angle variation between n^{th} probe angle φₙ and (n−1)^{th} Doppler angle correction value Δφₙ₋₁).

The processor 130 may be configured to calculate a corrected Doppler angle based on the Doppler angle correction value at step S308 in FIG. 3.

In one embodiment, the processor 130 may be configured to calculate the corrected Doppler angle as equation 6 provided below.

θ′ₙ=θ₀+Δφₙ (6)

In equation 6, θ′ₙ represents the corrected Doppler angle at the present cycle, and θ₀ represents an initial Doppler angle. The initial Doppler angle θ₀ may represent a Doppler angle that is initially set by the user or the ultrasound system.

In another embodiment, the processor 130 may be configured to calculate the corrected Doppler angle as equation 7 provided below.

θ′ₙ=θ′ₙ₋₁+Δφₙ (7)

In equation 7, θ′ₙ₋₁ represents the corrected Doppler angle at the previous cycle.

The processor 130 may be configured to calculate blood flow information (e.g., velocity of blood flow) at step S310 in FIG. 3. That is, the processor 130 may be configured to calculate the velocity of the blood flow ν by applying the corrected Doppler angle θ′ₙ to equation 3.

$$v = \frac{{cf}_{D}}{2 {f}_{0} cos {θ}_{n}^{′}} \qquad ( 8 )$$

The processor 130 may be configured to form the Doppler mode image based on the blood flow information (e.g., velocity of blood flow) at step S312 in FIG. 4. The methods of forming the Doppler mode image are well known in the art. Thus, they have not been described in detail so as not to unnecessarily obscure the present disclosure.

Optionally, the processor 130 may be configured to control the start and end of measuring the probe angle according to the Doppler angle correction start and the Doppler angle correction end by the user. Furthermore, the processor 130 may be configured to control the start and end of measuring the probe angle according to the start and end of acquiring of the second ultrasound data.

Referring back to FIG. 1, the memory 140 may store the ultrasound data acquired by the ultrasound data acquisition unit 120. The memory 140 may further store the Doppler angle calculated by the processor 130. The memory 140 may also store the Doppler angle correction value calculated by the processor 130.

The display unit 150 may be configured to display the B-mode image formed by the processor 130. The display unit 150 may be further configured to display the Doppler mode image formed by the processor 130. The display unit 150 may include a cathode ray tube (CRT) display, a liquid crystal display (LCD), an organic light emit diode (OLED) display and the like.

Any reference in this specification to “one embodiment,” “an embodiment,” “example embodiment,” “illustrative embodiment,” etc. means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to affect such feature, structure or characteristic in connection with other embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an inclination measuring unit configured to measure an inclination angle of an ultrasound probe at a predetermined cycle to form measuring information including the inclination angle, wherein the inclination measuring unit is mounted inside or outside of the ultrasound probe; and
a processing unit configured to calculate a Doppler angle correction value corresponding to the inclination angle based on the measuring information and calculate a corrected Doppler angle based on the Doppler angle correction value.

2. The ultrasound system of claim 1, wherein the processor is configured to calculate an angle variation between an initial inclination angle of the ultrasound probe and an inclination angle of the ultrasound probe at a preset cycle as the Doppler angle correction value based on the measuring information.

3. The ultrasound system of claim 2, wherein the processor is configured to calculate the corrected Doppler angle based on the Doppler angle correction value at the present cycle and an initial Doppler angle.

4. The ultrasound system of claim 1, wherein the processor is configured to calculate an angle variation between an inclination angle of the ultrasound probe at a present cycle and the an inclination angle of the ultrasound probe at a previous cycle as the Doppler angle correction value based on the measuring information.

5. The ultrasound system of claim 4, wherein the processor is configured to calculate the corrected Doppler angle based on the Doppler angle correction value at the present cycle and the corrected Doppler angle at the previous cycle.

6. A method of correcting a Doppler angle, comprising:
a) measuring an inclination angle of an ultrasound probe at a predetermined cycle to form measuring information including the inclination angle;
b) calculating a Doppler angle correction value corresponding to the inclination angle based on the measuring information; and
c) calculating a corrected Doppler angle based on the Doppler angle correction value.

7. The method of claim 6, wherein step b) comprises:
calculating an angle variation between an inclination angle of the ultrasound probe at a present cycle and an inclination angle of the ultrasound probe at a previous cycle as the Doppler angle correction value based on the measuring information.

8. The method of claim 7, wherein step c) comprises:
calculating the corrected Doppler angle based on the Doppler angle correction value at the present cycle and an initial Doppler angle.

9. The method of claim 6, wherein step b) comprises:
calculating an angle variation between an inclination angle of the ultrasound probe at a present cycle and an inclination angle of the ultrasound probe at a previous cycle as the Doppler angle correction value based on the measuring information.

10. The method of claim 9, wherein step c) comprises:
calculating the corrected Doppler angle based on the Doppler angle correction value at the present cycle and the corrected Doppler angle at the previous cycle.
